SEQUENCE LISTING (22 sequences)

SEQ ID NO: 1 - DNA, 20 bp, Homo sapiens
ctataagggactttccgctg

SEQ ID NO: 2 - DNA, 20 bp, Homo sapiens
atccgagcctgggagctctc

SEQ ID NO: 3 - DNA, 20 bp, Homo sapiens
atcagagcctgggagctctc

SEQ ID NO: 4 - DNA, 20 bp, Homo sapiens
tagatagccagagagctccc

SEQ ID NO: 5 - DNA, 20 bp, Homo sapiens
tttattgaggcttaagcagt

SEQ ID NO: 6 - DNA, 20 bp, Homo sapiens
ctttattgaggcttaagcag

SEQ ID NO: 7 - DNA, 20 bp, Homo sapiens
actcaaggcaagctttattg

SEQ ID NO: 8 - DNA, 20 bp, Homo sapiens
gcccgtctgttgtgtgactc

SEQ ID NO: 9 - DNA, 20 bp, Homo sapiens
accagagtcacacaacagac

SEQ ID NO: 10 - DNA, 20 bp, Homo sapiens
taccagagtcacacaacaga

SEQ ID NO: 11 - DNA, 20 bp, Homo sapiens
atcagatatccactgacctt

SEQ ID NO: 12 - DNA, 20 bp, Homo sapiens
ccggagtacttcaagaactg

SEQ ID NO: 13 - DNA, 20 bp, Homo sapiens
taacttgaagcaccatccaa

SEQ ID NO: 14 - DNA, 20 bp, Homo sapiens
gctgttggtctctcctttat

SEQ ID NO: 15 - DNA, 20 bp, Homo sapiens
ttacaccctgtgagcctgca

SEQ ID NO: 16 - DNA, 20 bp, Homo sapiens
cgtggcccgagagctgcatc

SEQ ID NO: 17 - DNA, 20 bp, Homo sapiens
ctgcaagggactttccgctg

SEQ ID NO: 18 - DNA, 20 bp, Homo sapiens
caagggaggtgtggcctggg

SEQ ID NO: 19 - DNA, 20 bp, Homo sapiens
agccgctttttgcctgtact

SEQ ID NO: 20 - DNA, 20 bp, Homo sapiens
atccgagcctgggagctctc

SEQ ID NO: 21 - DNA, 20 bp, Homo sapiens
tctcaaggcaagctttattg

SEQ ID NO: 22 - DNA, 20 bp, Homo sapiens
accagagtcacacaacagac

(19) United States
(12) Patent Application Publication
(10) Pub. No.: US 2018/0334732 A1
(43) Pub. Date: Nov. 22, 2018

(54) COMPOSITIONS AND METHODS FOR HIV QUASI-SPECIES EXCISION FROM HIV-1-INFECTED PATIENTS

(71) Applicant: DREXEL UNIVERSITY, Philadelphia, PA (US)

(72) Inventors: Brian WIGDAHL, Hummelstown, PA (US); Michael R. NONNEMACHER, Churchville, PA (US); William DAMPIER, Philadelphia, PA (US)

(21) Appl. No.: 15/529,337

(22) PCT Filed: Nov. 25, 2015
(86) PCT No.: PCT/US15/62755

§ 371 Date: May 24, 2017

Related U.S. Application Data
(60) Provisional application No. 62/084,182, filed on Nov. 25, 2014.

(51) Int. Cl. C12Q 1/70; C12Q 1/689

(52) U.S. Cl. CPC C12Q 1/70; C12Q 1/689

(57) ABSTRACT

The invention relates to the compositions and methods for complete excision of HIV-1 proviral genomes including viral quasi-species (vQS) from an HIV-1-infected human. The invention includes a composition of guide RNAs (gRNAs) designed to specifically target the HIV-1 LTR region or any other region of the HIV-1 genome. The invention further includes a method for treating an HIV-1-infected human using the clustered regularly interspaced short palindromic repeats (CRISPR)/CRISPR-associated (Cas) 9 system and the compositions of the present invention.

### CROSS REFERENCE TO RELATED APPLICATIONS

The present application claims priority under 35 U.S.C. § 119(e) to U.S. Provisional Patent Application No. 62/084,182, filed Nov. 25, 2014, which is incorporated herein by reference in its entirety.

### STATEMENT REGARDING FEDERALLY SPONSORED RESEARCH OR DEVELOPMENT

This invention was made with government support under NS032092, NS046263, and DA019807 awarded by National Institutes of Health. The government has certain rights in the invention.

### BACKGROUND OF THE INVENTION

More than 35 million people worldwide are currently reported to be infected with HIV-1, despite the currently adopted preventive and therapeutic measures. Furthermore, evolving HIV viral quasi-species (vQS) hamper development of a cure.

Patients who adhere to a highly active antiretroviral therapy (HAART) regimen typically maintain low or undetectable viral loads along with a near-normal CD4+ T-cell population. Reservoirs of latently infected hidden cells contain minimal levels of viral protein, and thus avoid both viral cytopathic effects and host immune clearance. The most prominent latently infected cell pool is thought to be the resting CD4+ memory T-cell compartment. However, in certain end-organs, such as the CNS, microglia and macrophages are likely the primary viral producer and reservoir. These reservoirs, particularly the CNS, are thought to be established shortly after the initial phase of infection.

The resting CD4+ memory T-cell population retains the capacity to produce infectious virus particles upon stimulation or cessation of HAART, and thus are a major barrier to achieving a definite HIV cure. Current efforts to eradicate HIV-1 from the resting CD4+ memory T-cell population primarily focus on a “shock and kill” method, where compounds are utilized to induce reactivation of virus from this cell population, whereby the host immune response recognizes and targets these infected cells. However, this type of therapeutic approach has several limitations. For instance, not all reservoirs with integrated provirus are (or can be) reactivated, thus leaving behind integrated HIV that may still be able to produce viral proteins or other components of the virus that could have adverse effects. Further, the cytotoxic T lymphocytes (CTLs) immune response is not robust enough to eliminate infected cells following reactivation.

Given these observations, current research objectives focus on reducing the size of the latently infected cell population without activating HIV gene expression or reactivating viral production. To date, four gene editing techniques have been examined within HIV eradication efforts: zinc finger nucleases (ZFN), transcription activator-like effector nucleases (TALENs), piggyback, and clustered regularly interspaced short palindromic repeats (CRISPR)/CRISPR-associated (Cas)9 system.

The CRISPR/Cas9 system was successfully used in genome engineering in yeast, Drosophila, human and mouse cell lines, and in zebrafish, mice, and C. elegans animal models. This technology has great promise for treating HIV infection. HIV targeting strategies include: disrupting HIV-1 entry coreceptors (CCR5, CXCR4) and proviral DNA-encoding viral proteins; engineering resistant cells by prior immunization with this type of therapeutic approach; selectively deleting HIV proviral DNA integrated into the host genome; and removing the proviral HIV-1 genome from host cell DNA, by targeting its highly-conserved 5′- and 3′-long terminal repeats (LTRs), or specific cis-acting elements within the LTR.

However, the CRISPR/Cas9 system has various complicating features. The system requires the design of a regimen of guide RNAs (gRNAs) that are complementary to the 5′- and 3′-ends of the desired excision site HIV's high mutability along with inter- and intra-patient variability make this a complicated problem. Further, gRNAs design principles are complex and limit the breadth of possible sequence targets. For instance, gRNAs require a match to the region of interest of an approximately twenty-nucleotide primer followed by the NGG protospacer adjacent motif (PAM) motif. The combination of these design principles with the variability of the vQS adds another layer of complexity for designing effective gRNAs.

There is a need in the art for identifying personalized and/or generalized methods for antiretroviral gene editing therapy in an HIV-infected patient. Such methods should eradicate any latent viral reservoirs in the patient, and treat and/or cure the HIV infection in the patient. The present invention satisfies this need.

### SUMMARY OF THE INVENTION

The invention provides a method of identifying one or more guide RNAs (gRNAs) that affect clustered regularly interspaced short palindromic repeats (CRISPR)/CRISPR associated (Cas)9 system-mediated cleavage of a viral genomic region that is chromosomally integrated in a subject infected with the virus, wherein the virus comprises one or more virus quasi-species (vQS). The invention further provides a tangible, non-transitory computer-readable medium comprising computer-executable instructions for implementing any of the methods of the invention.

In certain embodiments, the method comprises sequencing the test genomic material isolated from a bodily sample selected from the group consisting of a bodily sample of the virus-infected subject and bodily samples from a virus-infected patient population. In other embodiments, the method comprises identifying a given set of candidate gRNAs that aligns to the consensus sequence of the reference virus. In yet other embodiments, the method comprises comparing the given set of candidate gRNAs to the sequence of the test genomic material. In yet other embodiments, the methods of the invention allow for assessing whether each candidate gRNA in the given set affects CRISPR/Cas9-mediated cleavage of the viral genomic region that is chromosomally integrated in the virus-infected subject.

In certain embodiments, the virus comprises a lentivirus or retrovirus. In other embodiments, the virus comprises HIV-1 or HIV-2. In yet other embodiments, each one of the candidate gRNAs ends in GG. In yet other embodiments, the viral genomic region comprises an HIV-1 integrated long terminal repeat (LTR) region of at least one vQS. In yet other embodiments, the given set comprises only unique candidate gRNAs.

In certain embodiments, the method further comprises counting the number of individual instances of alignment between each candidate gRNA and the sequence of the test genomic material.

In certain embodiments, the method further comprises selecting a group of candidate gRNAs that have the highest number of individual instances of alignment for use in the comparing step, wherein the group comprises at least one gRNA.

In certain embodiments, the comparing step comprises applying a binding matrix. In other embodiments, the binding matrix assigns a position-specific penalty for a mismatch between each candidate gRNA and the sequence of the test genomic material.

In certain embodiments, the identifying step compares only positions of the consensus sequence ending in GG.

In certain embodiments, the comparing step comprises utilizing a statistical OR function.

In certain embodiments, the method further comprises applying a numerical optimization technique to select one or more effective candidate gRNAs.

The invention further provides a method of treating HIV-1 infection in an infected human.

In certain embodiments, the method comprises sequencing HIV-1 long terminal repeat (LTR) regions that are integrated in the human genomic DNA from a sample selected from the group consisting of a bodily sample from the HIV-1-infected human or bodily samples from a HIV-1-infected patient population. In other embodiments, the method further comprises identifying a set of guide RNA sequences (gRNAs) that are at least partially identical to a fragment of the HIV-1 LTR regions. In yet other embodiments, the method comprises excising the HIV-1 chromosomally integrated genome from the human genomic DNA of the human using the set of gRNAs and the clustered regularly interspaced short palindromic repeats (CRISPR)/CRISPR associated (Cas)9 system.

In certain embodiments, the sequencing comprises next-generation sequencing. In other embodiments, sequencing is performed using long fragment polymerase chain reaction (PCR) with fragments ranging from about 50 to about 10,000 base pairs. In yet other embodiments, the fragment of the HIV-1 LTR regions is selected from at least one from the group consisting of the 5′-end and 3′-end. In yet other embodiments, the human is infected with one or more evolving HIV-1 quasi-species (vQS). In yet other embodiments, the vQS comprise one or more viral single nucleotide polymorphism (vSNP) in the integrated HIV-1 LTR nucleotide sequence, as compared to the HIV-1 LTR nucleotide sequence from an HIV-1 reference/consensus strain. In yet other embodiments, the one or more HIV-1 vQS latently infect at least one host cell selected from the group consisting of a macrophage, gut-associated lymphoid cell, microglial cell, astrocyte, and resting CD4+ memory T-cell. In yet other embodiments, the gRNAs are at least partially identical to the 5′- and 3′-ends of the HIV-1 vQS LTR regions. In yet other embodiments, the gRNAs are identical to the 5′- and 3′-ends of the HIV-1 vQS LTR regions.

In certain embodiments, the human is being administered HAART.

In certain embodiments, the sequencing is repeated at two or more time points, and the number of HIV-1 vQS infecting the human is estimated at each time point. In other embodiments, the excision step is performed when the number of vQS is minimized or has reached a minimum over the sequencing time period. In yet other embodiments, the number of gRNAs in the set ranges from about 1 to about 100.

In certain embodiments, the excision step comprises administering to the human the set of gRNAs within at least one selected from the group consisting of a viral vector, microparticle, nanoparticle, liposome, hydrogel, and block copolymer micelle. In other embodiments, the viral vector is at least one selected from the group consisting of a lentiviral vector, retroviral vector, adenoviral vector, and adeno-associated viral (AAV) vector.

In certain embodiments, the vSNP is present in at least one nucleotide position selected from the group consisting of 1-800. In other embodiments, the HIV-1 consensus strain is from the subtype B. In yet other embodiments, the human genomic DNA is extracted from a sample selected from the group consisting of blood, lymphoid tissue, bone marrow, plasma sample, peripheral blood mononucleated cell (PBMC) and CD4+ memory T-cell.

The invention further provides a method of treating HIV-1 infection in an infected human.

In certain embodiments, the method comprises obtaining a set of guide RNA sequences (gRNAs) that are at least partially identical to a fragment of the HIV-1 LTR regions. In other embodiments, the method comprises excising the HIV-1 genome from the genomic DNA of the human using the set of gRNAs targeted to the HIV-1 LTR or another region of the HIV-1 and the CRISPR-Cas9 system.

The invention further provides an isolated set of guide RNAs (gRNAs), the set comprising gRNAs that are at least partially identical to a fragment of the HIV-1 LTR regions that are integrated in the genomic DNA of an HIV-1-infected human.

In certain embodiments, the fragment of the HIV-1 LTR regions is selected from at least one from the group consisting of the 5′-end and 3′-end. In other embodiments, the human is infected with one or more HIV-1 quasi-species (vQS). In yet other embodiments, the vQS comprise one or more viral single nucleotide polymorphism (vSNP) in the integrated HIV-1 LTR nucleotide sequence, as compared to the HIV-1 LTR nucleotide sequence from an HIV-1 reference/consensus strain. In yet other embodiments, the HIV-1 consensus strain is from the subtype B. In yet other embodiments, the HIV-1 vQS latently infect at least one host cell selected from the group consisting of a macrophage, gut-associated lymphoid cell, microglial cell, astrocyte, and resting CD4+ memory T-cell. In yet other embodiments, the gRNAs are at least partially identical to the 5′- and 3′-ends of the HIV-1 vQS LTR regions. In yet other embodiments, the gRNAs are identical to the 5′- and 3′-ends of the HIV-1 vQS LTR regions. In yet other embodiments, the number of gRNAs in the set ranges from about 1 to about 100. In yet other embodiments, the set is packaged in at least one selected from the group consisting of a viral vector, microparticle, nanoparticle, liposome, hydrogel, and block copolymer micelle. In yet other embodiments, the viral vector is at least one selected from the group consisting of a lentiviral vector, retroviral vector, adenoviral vector, and adeno-associated viral (AAV) vector. In yet other embodiments, the vSNP is present at the nucleotide position selected from the group consisting of 1-800. In yet other embodiments, the isolated set comprises at least one gRNA encoded by a DNA sequence selected from the group consisting of SEQ ID NOs:1-10 and SEQ ID NOs:13-22. In yet other embodiments, the isolated set comprises each gRNA encoded by the DNA sequences from the group consisting of SEQ ID NOs:1-10. In yet other embodiments, the isolated set comprises each gRNA encoded by the DNA sequences from the group consisting of SEQ ID NOs:13-22.

The invention further provides a method of defining an HIV population or identifying to which HIV population a human subject belongs. In certain embodiments, the method comprises obtaining a DNA sequencing file for the human subject, wherein the file comprises an ordered sequence of bases corresponding to the subject's DNA. In other embodiments, the method comprises using an alignment program to align the human subject's DNA sequence with a reference sequence, whereby the program generates a computer-readable VCF (Variant Call Format) file indicating regions of alignment. In yet other embodiments, the method comprises analyzing differences of regions of human subject's DNA as compared to the reference sequence. In yet other embodiments, the analysis allows for defining an HIV population or identifying to which HIV population a human subject belongs.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1E are a series of graphs illustrating HIV LTR genetic variation and gRNA design in well-controlled patients.

FIG. 1A is a set of graphs illustrating mutations per LTR per year. LTRs from 21 patients naïve to ART (green lines), 39 patients off/non-adherent ART (red lines), 168 patients on/adherent ART (grey lines), and 54 patients on/adherent ART (black lines) with viral loads (VL) always below 100 copies/ml with multiple visits were compared longitudinally by individually aligning all sequences from each patient and calculating the number of mutations between consecutive visits. The number of nucleotide changes per 100 bp was plotted against the time since the baseline visit, to determine the rate of accumulated variations. The top panel depicts all longitudinal samples per patient. The bottom panel shows median and standard deviation for each group at each year.

FIG. 1B is a set of graphs illustrating mutations per LTR per year. LTRs from 45 patients on/adherent ART and 31 patients discontinuous with ART with at least three visits were analyzed as in FIG. 1A. The trajectory of each patient is shown in narrow lines with the median and the upper and lower quartiles in bold lines.

FIG. 1C is a table illustrating PIDs, position and minimum gRNAs. Utilizing Roche 454 next generation sequencing (NGS), NGS on genomic DNA isolated from PBMCs of 6 patients and 8 samples was performed on a 4.4 kb fragment of the HIV genome (Li, et al., 2011, J. Neurovirol. 17 (1):92-109). The number of gRNAs and target position for each patient sequenced was determined by using the CRISPR design tool described in Hu, et al., 2004, PNAS U S A 111 (31):11461-6, for all 454 reads and then checked for homology across the deep sequencing data.

FIG. 1D is a graph wherein NGS reads from patient 17 visit 3 were mapped to HXB2 and examined for percent conservation (top line) and number of gRNAs necessary for excision of all known quasi-species (bottom line) at every position of the LTR.

FIG. 1E is a table summarizing the minimum number of gRNAs at a given HIV-1 LTR position.

FIG. 2 is a series of graphs illustrating gRNA design and the number of cuts per sample for three different gRNA packages across three different datasets. The “All” dataset (left panel) includes all NGS data currently performed by the team (N=269), the “Testing” dataset (middle panel) contains a subset of these data (N=169), and the “NNTC” dataset (right panel) is a set of patients from an independent cohort of samples (N=5).

FIG. 3 is a boxplot showing the spread of gRNA cleavage likelihoods of gRNAs A and B on the Drexel Medicine CARES cohort patient samples. The box indicates the inner quartile ranges, the whiskers indicate the 95% confidence intervals, and the line indicates the mean value of the percentage of the vQS that are cleaved by the gRNAs.

FIG. 4 is a series of graphs illustrating the effectiveness of previously described gRNAs compared to the package of gRNAs devised by methods described herein for samples from subtype B from all of North America.

FIG. 5 is a table showing the packages of gRNAs selected using three different methodologies (the gRNAs are represented by the DNA sequences that encode them). The Temple package was selected using previously described methods. The Top-10 gRNAs were selected as those with the highest average cleavage score across 100 patients in the training dataset. The SMRT-10 package was selected using numerical optimization from the same dataset. The values reported are the fraction of samples in each dataset that have a greater than 80% likelihood of being cleaved.

### DETAILED DESCRIPTION OF THE INVENTION

The invention relates to the unexpected discovery of composition and methods for complete and/or therapeutically efficacious excision of HIV-1 proviral genomes including viral quasi-species (vQS) from an infected human's genome. The invention includes a composition comprising guide RNAs (gRNAs) that are identical or quasi-identical to the HIV-1 LTR region or other viral genomic region. In certain embodiments, the gRNAs are specific to one or more vQS that infect the human. In other embodiments, the gRNAs are specific to one or more vQS present in an HIV-1-infected patient population.

The invention further includes a method for treating an HIV-1-infected human using the clustered regularly interspaced short palindromic repeats (CRISPR)/CRISPR-associated (Cas) 9 system. In certain embodiments, the method comprises administering to the human a composition of gRNAs specific to one or more vQS that infect the human. In other embodiments, the method comprises administering to the human a composition comprising gRNAs specific to one or more vQS present in an HIV-1-infected patient population. In yet other embodiments, the composition is delivered to the human using any available gene delivery system, such as but not limited to microparticles, nanoparticles, liposomes, hydrogels, or block copolymer micelles.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, specific materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein, when referring to a measurable value such as an amount, a temporal duration, and the like, the term “about” is meant to encompass variations of ±20% or ±10%, more specifically ±5%, even more specifically ±1%, and still more specifically ±0.1% from the specified value, as such variations are appropriate to perform the disclosed methods.

As used herein, the term “aggregated” refers to any methodology that summarizes multiple numbers into a single number describing certain aspect(s) of the collection. In certain embodiments, this is a statistical function exemplified by a mean, median, and/or mode. In other embodiments, outlier values are excluded.

As used herein, the terms “alignment” and “sequence alignment”, which are used interchangeably, refer to any type of algorithm that finds the ideal correspondence between two or more sequences. This can be done with certain computational tools common to one skilled in the art. In certain embodiments, the alignment can be further refined with human intervention. In other embodiments, the alignment can be further refined without human intervention.

As used herein, the term “amount” refers to the abundance or quantity of a constituent in a mixture.

As used herein, the term “amplicon” or “PCR products” or “PCR fragments” or “amplification” products refers to extension products that comprise the primer and the newly synthesized copies of the target sequences.

As used herein, the term “antiretroviral” as applied to an agent, drug, preparation, composition or the like refers to an agent, preparation, composition or the like that controls or inhibits the proliferation or multiplication of a retrovirus in a host that is susceptible to the retrovirus.

“Antiretroviral therapy” or “antiretroviral drug” are used interchangeably herein to refer to a nucleoside reverse transcriptase inhibitor, an entry inhibitor, an integrase inhibitor, a fusion inhibitor, a protease inhibitor, and/or a non-nucleoside reverse transcriptase inhibitor, collectively known as HAART. Such antiretroviral therapy regimens include, but are not limited to, one or a combination of the following drugs: COMBIVIR® (lamivudine and zidovudine), EMTRIVA® (FTC, emtricitabine), EPIVIR® (lamivudine, 3TC), HIVID® (zalcitabine, ddC, dideoxycitidine), RETROVIR® (zidovudine, AZT, azidothymidine, ZDV), TRIZIVIR® (abacavir, zidovudine, lamivudine), VIDEX® (didanosine, ddI, dideoxyinosine), VIDEX® EC (enteric coated didanosine), VIREAD® (tenofovir disoproxil fumarate), ZERIT® (stavudine, d4T), ZIAGEN® (abacavir), AGENERASE® (amprenavir), CRIXIVAN® (indinavir, IDV, MK-639), FORTOVASE® (saquinavir), INVIRASE® (saquinavir mesylate, SQV), KALETRA® (lopinavir and ritonavir), NORVIR® (ritonavir, ABT-538), REYATAZ® (atazanavir sulfate), VIRACEPT® (nelfinavir mesylate, NFV), FUZEON® (enfuvirtide, T-20), RESCRIPTOR® (delavirdine, DLV), SUSTIVA® (efavirenz) and VIRAMUNE® (nevirapine, BI-RG-587).

As used herein, the term “binding matrix” refers to a mathematical representation of the ability of the guide sequence to facilitate the action of the recruited protein. In certain embodiments, the matrix comprises a list of position-dependent penalties for non-complementarity between the guide sequence and its target. In other embodiments, the matrix comprises a regular expression, which matches a particular sequence of nucleotides. In yet other embodiments, the matrix comprises a log-odds score of having a particular nucleotide at a particular position. In yet other embodiments, the matrix comprises an arbitrarily defined mathematical or statistical function that compares two or more potential sequences and yields a likelihood of effect.

The term “biomarker” or “marker” as used herein refers to molecules, such as a polynucleotide or a polypeptide, in an individual that are differentially present (i.e., present in increased or decreased levels) depending on presence or absence of a certain condition, disease, or complication. In certain embodiments, biochemical markers are gene expression products that are differentially present (e.g., through increased or decreased level of expression or turnover) in presence or absence of a certain condition, disease, or complication. The level of a suitable biomarker can indicate the presence or absence of a particular condition, disease, or risk, and thus allow diagnosis or determination of the condition, disease or risk.

As used herein, the term “bp” refers to base pair.

As used herein, “CD4+ subclass of T-lymphocytes” (or “CD4+ T cells”) are white blood cells that are an essential part of the human immune system, particularly in the adaptive immune system. CD4+ T cells play a critical role in maintaining CTL function in viral infection, and are the primary target of HIV infection. When a quantitative decline in the number of CD4+ lymphocytes and a qualitative impairment of their function are observed in HIV-1-infected patients, these patients progress into acquired immunodeficiency syndrome (AIDS) with a higher chances to develop opportunistic infections, neurological disease, neoplastic growth and/or eventual death. As established by the Centers for Disease Control and Prevention (CDC), a person with HIV and a CD4 count below 200 or a CD4 percentage below 14% is considered to have AIDS. A CD4 test quantifies Helper T cells and is often combined with viral load testing to monitor the progression of HIV. A decreased CD4 count, in combination with higher numbers on a viral load test, indicates an increased risk of contracting opportunistic infections. However, a patient can recover HIV-specific CD4+ T-cell response, for example after being administered the highly active anti-retroviral therapy (HAART).

As referred to herein, a “computational pipeline” refers to a collection of functions that are arranged such that the output of one or more functions, often a set of analyzed computer files, are provided as inputs to another function or functions. In certain embodiments, these require information from the user and in some cases they are completely autonomous.

The term “concentration” refers to the abundance of a constituent divided by the total volume of a mixture. The term concentration can be applied to any kind of chemical mixture, but most frequently it refers to solutes and solvents in solutions.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, a “function” refers to any computational program that accepts a set of input data and produces a set of output data.

As used herein, “guide RNAs”, “guiding RNAs” or “gRNAs” refer to a set of nucleotides that are complementary to a target DNA sequence (on average about 16, 18, 20, 22 or 24 nucleotides) and that direct certain nucleases (RNA-guided nucleases, RGNs) to specific genomic loci for gene-editing purposes.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV (human), SIV (simian), BIV (bovine) and FIV (feline) are all examples of lentiviruses. Vectors derived from lentiviruses offer the means to achieve significant levels of gene transfer in vivo and ex vivo.

The term “measuring” as used herein relates to determining the amount or concentration, preferably semi-quantitatively or quantitatively. Measuring can be done directly and/or indirectly.

A “mutation” as used therein is a change in a DNA sequence resulting in an alteration from a given reference sequence (which may be, for example, an earlier collected DNA sample from the same subject). The mutation can comprise deletion and/or insertion and/or duplication and/or substitution of at least one deoxyribonucleic acid base such as a purine (adenine and/or thymine) and/or a pyrimidine (guanine and/or cytosine). Mutations may or may not produce discernible changes in the observable characteristics (phenotype) of an organism (subject).

As used herein, a “nanoparticle” is a particle ranging in size from about between 1 and about 1,000 nanometers, and any interval thereinbetween. In certain embodiments, nanoparticles can be used for drug delivery. In other embodiments, nanoparticles can be used to deliver nucleic acids to the interior of a cell.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

The term “numerical optimization” refers to a broad term for a class of algorithms that optimize the value of a function by changing the input parameters. In certain embodiments, one tries all possible inputs. In other embodiments, derivatives are calculated between the input values and the output values, that could be used to guide the selection of the inputs to try next. In yet other embodiments, biological and physical processes are modeled to guide the selection of inputs; this includes, but is not limited to, genetic algorithms, simulated annealing, and ant-colony optimization algorithms.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, one skilled in the art “obtains” an experimental result, data set, material, conclusion or any other piece of knowledge when one comes into possession of such experimental result, data set, material, conclusion or any other piece of knowledge, which may have been acquired by one or more third parties or by the one skilled in the art in its entirety or at least partially. In certain embodiments, one skilled in the art obtains experimental data, which may be raw data or at least partially processed data, and processes and/or manipulates the data as to reach at least one scientific conclusion or inference. In other embodiments, one skilled in the art obtains at least one scientific conclusion or inference that is derived from experimental data by one or more third parties' processing and/or manipulation. In other embodiments, one skilled in the art obtains at least one material that is identified and/or prepared by one or more third parties.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polynucleotide” includes cDNA, RNA, DNA/RNA hybrid, anti-sense RNA, siRNA, miRNA, snoRNA, genomic DNA, synthetic forms, and mixed polymers, both sense and antisense strands, and may be chemically or biochemically modified to contain non-natural or derivatized, synthetic, or semisynthetic nucleotide bases. Also, included within the scope of the invention are alterations of a wild type or synthetic gene, including but not limited to deletion, insertion, substitution of one or more nucleotides, or fusion to other polynucleotide sequences.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

A “primer” is an oligonucleotide, usually of about 15, 20, 25, 30, 35, 40, 45 or 50 nucleotides in length, that is capable of hybridizing in a sequence specific fashion to the target sequence and being extended during the PCR.

As used herein, a “program” refers to a computational tool that is used, in an unaltered form, by a user. These tools often perform a limited range of functions and are intended to be linked with other programs to accomplish a task none of them can complete individually.

As used herein, the term “read mapping” is the process of finding the optimal location of a small sequence segment on the genome. This is often done using computational tools termed “mappers” or “short-read aligners”, terms which are used interchangeably.

As used herein, the terms “reference” or “control” are used interchangeably, and refer to a value that is used as a standard of comparison (e.g., average CD4+ T cell count or viral Load in HIV-1-infected subjects; a level of gene expression in the HIV-1 consensus strain).

As used herein, the term “reference alignment” is used to specifically refer to an alignment in which the query sequence is aligned to a known HIV strain commonly used for this purpose.

As used herein, the term “retrovirus” refers to a single-stranded RNA virus that converts its RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants.

The term “RNA” as used herein is defined as ribonucleic acid.

A “sample” or “biological sample” as used herein means a biological material from a subject, including but is not limited to organ, tissue, exosome, blood, plasma, saliva, urine and other body fluid. A sample can be any source of material obtained from a subject.

A “single nucleotide polymorphism” (SNP), as referred herein, represents a variation in one or more single nucleotide changes in a DNA sequence among organisms, such among viruses, among mammals, or among humans. For instance, a SNP may replace the nucleotide cytosine (C) with the nucleotide thymine (T) in a certain stretch of DNA. SNPs are the most common type of genetic variation among people and occur normally throughout a person's DNA (around 10 million SNPs in the human genome). Most commonly, these variations are found in the non-coding DNA between genes. They can act as biological markers and can be associated with certain diseases particularly when they occur within a gene or in a regulatory region near a gene. In the cases where SNPs occur within a gene, they may lead to variations in the amino acid sequence. SNPs can help predicting an individual's response to certain drugs, susceptibility to environmental factors such as toxins, and risk of developing particular diseases.

A “subject” or “patient” as used therein may be a human or non-human mammal. Non-human mammals include, for example, livestock and pets, such as ovine, bovine, porcine, canine, feline and murine mammals. In certain embodiments, the subject is human.

The term “therapeutic” as used herein means a treatment and/or prophylaxis. A therapeutic effect is obtained by suppression, remission, or eradication of a disease state.

As used herein, to “treat” means reducing the frequency with which symptoms of a disease, disorder, or adverse condition, and the like, are experienced by a subject.

The term “treatment” as used within the context of the present invention is meant to include therapeutic treatment as well as prophylactic, or suppressive measures for the disease or disorder. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

A “vector” is a composition of matter comprising an isolated nucleic acid, and can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

The term “viral quasi-species” or “vQS” refers to a collection of viral sequences from the same patient.

As used herein, “10% greater” refers to expression levels that are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween, than a control or a reference.

As used herein, “10% lower” refers to expression levels that are at least 10% or more, for example, 20%, 30%, 40%, or 50%, 60%, 70%, 80%, 90% lower or more, and/or 1.1 fold, 1.2 fold, 1.4 fold, 1.6 fold, 1.8 fold, 2.0 fold lower or more, and any and all whole or partial increments therebetween, than a control or a reference.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention relates to the unexpected discovery of compositions and methods for complete and/or therapeutically effective excision of HIV-1 proviral genomes, including viral quasi-species (vQS), from an infected human's genome.

The invention further includes an in silico method for estimating the fraction of vQS that is affected by CRISPR directed cleavage utilizing specific guide RNAs (gRNAs) that are identical or quasi-identical to the HIV-1 LTR region or any other viral genomic region derived from the chromosomally integrated provirus from a human and/or an HIV-1-infected patient population. In certain embodiments, the method comprises using a binding matrix and applying it to all sequences gathered using a next generation sequencing platform. In other embodiments, the output of the binding matrix is then aggregated over all sequences gathered from the biological sample(s).

The invention further includes a method for treating an HIV-1-infected human using the clustered regularly interspaced short palindromic repeats (CRISPR)/CRISPR-associated (Cas) 9 system. In certain embodiments, the method comprises administering to the human a composition of gRNAs specific to one or more vQS that infect the human. In other embodiments, the method comprises administering to the human a composition of gRNAs specific to one or more vQS that infect an HIV-1-infected patient population.

The present invention, using the Drexel Medicine CNS AIDS Research and Eradication Study (CARES) Cohort, reveals key effects of variation within the viral genome. The variation assessed herein occurred within patients regardless of HAART status, demonstrating that viral replication and further variation within the viral genome continue even in patients with effective therapeutic management.

This invention represents a unique approach involving the use of specific LTR vSNPs to define sets of guide RNAs specific for viral quasi-species. The guide RNAs of this invention, in conjunction with CRISPR/Cas9, create an ideal system for excising HIV-1 integrated genome and thus are powerful HIV-1 curative tools.

In certain embodiments, the present invention provides a method of treating an HIV-1-infected human by excising the HIV-1 proviral genomes, including viral quasi-species (vQS), that are integrated in the genomic DNA of the HIV-1-infected human.

The method comprises sequencing at least a portion of the genomic DNA contained in a bodily sample taken from an HIV-1-infected patient and/or bodily samples taken from an HIV-1-infected patient population. In certain embodiments, the sample comprises blood, serum, plasma or other bodily fluids, such as interstitial fluid, urine, whole blood, saliva, serum, lymph, gastric juices, bile, sweat, tear fluid, brain and/or spinal fluids, and bone marrow. In other embodiments, the sample comprises any type of tissue, including but not limited to lymphoid tissues, such as but not limited to reticular cells, white blood cells (such as macrophages and/or leukocytes), bone marrow, thymus, spleen, mucosa-associated lymphoid tissues, and lymph nodes. In yet another embodiment, the sample contains any types of cells, including but not limited to white blood cells.

In certain embodiments, the bodily sample is processed and/or manipulated prior to being analyzed. In other embodiments, the bodily sample is not processed and/or manipulated prior to being analyzed. In yet other embodiments, the sample is derived from a tissue culture or from the supernatant media of a tissue culture, wherein the tissue culture comprises material taken from the patient.

The genomic DNA (gDNA) may be extracted from the patient's bodily sample, allowing for the analysis of the HIV-1 integrated long terminal repeat (LTR) region therein. In certain embodiments, the HIV-1 integrated LTR region is selectively amplified and sequenced using methods known in the art. For instance, the viral LTR region can be amplified by polymerase chain reaction (PCR) and sequenced thereafter. In certain embodiments, the LTR region comprises a sequence within the U3, R and/or U5 regions.

The “polymerase chain reaction” (also referred to as “PCR”) is a reaction in which replicate copies are made of a target polynucleotide using a “pair of primers” or “set of primers” comprising a “upstream” primer and a “downstream” primer, and a catalyst of polymerization, such as a DNA polymerase, which is typically a thermally-stable polymerase enzyme. Methods for PCR are known in the art (“PCR”, Ed. M. J. McPherson and S. G Moller (2000) BIOS Scientific Publishers Ltd, Oxford). PCR can be performed on cDNA obtained from reverse transcribing mRNA isolated from biological samples. In certain embodiments, in order to circumvent a biased introduction of unnatural viral nucleotide variation, a high fidelity polymerase is selected. Non limiting examples include the PHUSION® DNA polymerase, iPROOF® DNA polymerase, PLATINUM® DNA polymerase, and variations or analogues thereof.

In certain embodiments, other nucleic acid amplification techniques are used to analyze the sample. One such method for amplification is reverse transcription polymerase chain reaction (RT-PCR). First, complementary DNA (cDNA) is made from an RNA template, using a reverse transcriptase enzyme, and then PCR is performed on the resultant cDNA.

Another method for amplification is the ligase chain reaction (“LCR”), disclosed in EP 0 320 308, incorporated herein in its entirety by reference. In LCR, two complementary probe pairs are prepared, and in the presence of the target sequence, each pair binds to opposite complementary strands of the target such that they abut. In the presence of a ligase, the two probe pairs link to form a single unit. By temperature cycling, as in PCR, bound ligated units dissociate from the target and then serve as “target sequences” for ligation of excess probe pairs. U.S. Pat. No. 4,883,750, incorporated herein in its entirety by reference, describes a method similar to LCR for binding probe pairs to a target sequence.

Various sequencing platforms are known in the art, and the choice of a particular platform may be based on the user's and experiment's requirements. In certain embodiments, the sequencing method is a basic chain-termination method. In other embodiments, the sequencing method is a more advanced high throughput next-generation method. Non limiting example of massively parallel signature sequencing platforms are Illumina sequencing by synthesis (Illumina, San Diego, Calif.), 454 pyrosequencing (Roche Diagnostics, Indianapolis Ind.), SOLiD sequencing (Life Technologies, Carlsbad, Calif.), Ion Torrent semiconductor sequencing (Life Technologies, Carlsbad, Calif.), Heliscope single molecule sequencing (Helicos Biosciences, Cambridge, Mass.), and single molecule real time (SMRT) sequencing (Pacific Biosciences, Menlo Park, Calif.). In yet other embodiments, the next generation sequencing utilizes long fragment polymerase chain reaction (PCR) with varying ranges of base pairs (bp). In certain embodiments, the fragments range from 30 to about 300 bp, or from 50 to about 100 bp.

Sequence reads from these tools often contain errors and must be processed to remove those errors that would contaminate downstream results. This can be done by using common tools that look at parameters including, but not limited to, average quality of the read, length of the read, and the presence of long runs of the same nucleotide. These tools can be calibrated with control experiments, or they can work without such controls.

Filtered, or in some-cases unfiltered, sequence reads are processed to determine the sequence and quantity of each vQS contained within the sample. Computational algorithms designed to solve this problem are known to those skilled in the field. In certain embodiments, these algorithms are encapsulated in other programs. In other embodiments, they are implemented by the inventor. These algorithms are arranged into a computational pipeline so they can be done consistently across an unlimited number of users without human error.

In certain embodiments, the sequenced HIV-1 integrated LTR region invention is aligned and compared to a HIV consensus reference genomic region. In other embodiments, the sequencing information is analyzed using a computational method. In yet other embodiments, the sequences are aligned to the HIV-1 genome using the BWA alignment tool. However, a wide number of computational methods that are applicable to the methods of the invention are appreciated and performed by those skilled in the art.

The methods described herein can be readily implemented in software that can be stored in computer-readable media for execution by a computer processor. For example, the computer-readable media can be volatile memory (e.g., random access memory and the like) non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

In certain embodiments, the HIV-1 consensus strain belongs to any group and subtype known in the art. Briefly, HIV-1 is genotypically divided into three distinctive groups: M, N, and O. Group M comprises most of the HIV-1 strains that cause acquired immune deficiency syndrome (AIDS) worldwide and has been further subdivided into 9 different subtypes (A-D, F-H, J and K). In other embodiments, the HIV-1 consensus strain is from the sub-type B. In other embodiments, the invention further includes HIV-2 and other retroviruses and lentiviruses.

In certain embodiments, the presence of a nucleotide variation (or a SNP) of at least one nucleotide in the tested sample relative to the consensus sample indicates that the patient is subject to increased rate of disease progression and/or severity. In other embodiments, the presence of a SNP is indicative of a change in the patient's immune response. In certain embodiments, the detection of one or more vSNP in the integrated HIV-1 LTR nucleotide sequence from a patient is indicative of the HIV-1 disease stage in the periphery and the brain of the patient. HIV-1 infection can be associated with neurocognitive impairment. The blood-brain-barrier (e.g., leukocytes, and mainly monocytes/macrophages from the periphery) plays a critical role in neuroinvasion. Certain specific HIV-1 subtypes are known to associate with a higher incidence of brain phenotype and/or HIV-associated dementia (HAD).

In certain embodiments, the HIV-1 infecting strains evolve through time into HIV-1 quasi-species (vQS). In other embodiments, the vQS comprise one or more viral single nucleotide polymorphism (vSNP). In yet other embodiments, the vQS are latent in the host macrophage, gut-associated lymphoid cell, microglial cell, astrocyte, and/or resting CD4+ memory T-cell.

In certain embodiments, the sequencing of the patient's DNA is repeated at two or more time points. In other embodiments, the comparison of the patient's sequencing results at various time points allows for estimating the number of HIV-1 quasi-species (vQS) infecting the human at the various time points.

In certain embodiments, the chromosomally integrated HIV-1 genome is excised by a gene-editing technology. Several gene editing technologies are known in the art and can be used for HIV eradication. Non limiting examples include zinc finger nucleases (ZFN), transcription activator-like effector nucleases (TALENs), piggyback, and clustered regularly interspaced short palindromic repeats (CRISPR)/CRISPR-associated (Cas) system. In certain embodiments, the invention utilizes the CRISPR/Cas 9 system. In yet other embodiments, the excision is performed in vivo and/or in vitro. In yet other embodiments, the excision is performed in a cultured cell, and the cultured cell is then reintroduced in the subject.

In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino, et al., 1987, J. Bacteriol. 169:5429-5433; Nakata, et al., 1989, J. Bacteriol. 171:3553-3556), and associated genes. Similar interspersed SSRs have been identified in other bacteria (Groenen, et al., 1993, Mol. Microbiol. 10:1057-1065; Hoe, et al., 1999, Emerg. Infect. Dis. 5:254-263; Masepohl, et al., 1996, Biochim. Biophys. Acta 1307:26-30; Mojica, et al., 1995, Mol. Microbiol. 17:85-93). The CRISPR loci typically differ from other SSRs by the structure of the repeats, which have been termed short regularly spaced repeats (SRSRs) (Janssen, et al., 2002, OMICS J. Integ. Biol. 6:23-33; Mojica, et al., 2000, Mol. Microbiol. 36:244-246). In general, the repeats are short elements that occur in clusters that are regularly spaced by unique intervening sequences with a substantially constant length. Although the repeat sequences are highly conserved between strains, the number of interspersed repeats and the sequences of the spacer regions typically differ from strain to strain (van Embden, et al., 2000, J. Bacteriol. 182:2393-2401). CRISPR loci have been identified in more than 40 prokaryotes (Jansen, et al., 2002, Mol. Microbiol. 43:1565-1575).

In general, “CRISPR system” refers collectively to transcripts and other elements involved expressing, or directing the activity of, CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, complete complementarity is not needed, provided this is sufficient to be functional. In certain embodiments, the tracr sequence has at least 50%, 60%, 70%, 80%, 90%, 95% or 99% of sequence complementarity along the length of the tracr mate sequence when optimally aligned. In other embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′- with respect to (“upstream” of) or 3′- with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In general, a guide sequence is any polynucleotide sequence having sufficient complementarity with a target polynucleotide sequence to hybridize with the target sequence and direct sequence-specific binding of a CRISPR complex to the target sequence. In certain embodiments, the degree of complementarity between a guide sequence and its corresponding target sequence, when optimally aligned using a suitable alignment algorithm, is at least about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (maq dot sourceforge dot net). In certain embodiments, a guide sequence is at least about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In other embodiments, a guide sequence is less than about 75, 50, 45, 40, 35, 30, 25, 20, 15, 12, or fewer nucleotides in length. In yet other embodiments, a guide sequence is about 23 nucleotides in length.

The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex (including the guide sequence to be tested and a control guide sequence different from the test guide sequence), and comparing binding or rate of cleavage at the target sequence between the test and control guide sequence reactions. Other assays are contemplated by those skilled in the art. A guide sequence may be selected to target any target sequence. In certain embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMNNNNNNNNNNNXGG where NNNNNNNNNNNNXGG (N is A, G, T, or C; and X can be anything) has a single occurrence in the genome.

In certain embodiments, the guide sequence is a guide RNA (gRNA, or guiding RNA). The gRNA interacts with the CRISPR/Cas to guide it to a specific target site, wherein the effector domain of the CRISPR/Cas modifies the chromosomal sequence or regulates expression of the chromosomal sequence. Each guide RNA comprises three regions: a first region at the 5′-end that is complementary to the target site in the chromosomal sequence, a second internal region that forms a stem loop structure, and a third 3′-region that remains essentially single-stranded.

The first region of each guide RNA is distinct, such that each guide RNA guides a fusion protein to a specific target site. The second and third regions of each guide RNA can be the same in all guide RNAs. The first region of the guide RNA is complementary to the target site in the chromosomal sequence, such that the first region of the guide RNA can base pair with the target site. In certain embodiments, the first region of the guide RNA can comprise from about 10 nucleotides to more than about 25 nucleotides. For example, the region of base pairing between the first region of the guide RNA and the target site in the chromosomal sequence can be about 10, 11, 12, 13, 14, 15, 16, 17, 18, 19, 20, 22, 23, 24, 25, or more than 25 nucleotides in length. In other embodiments, the first region of the guide RNA is about 20 nucleotides in length. In yet other embodiments, the first region of the guide RNA is about 18 nucleotides in length. In yet other embodiments, the first region of the gRNAs are at least partially identical to the 5′- and 3′-ends of the HIV-1 LTR regions. In yet other embodiments, the first region of the gRNAs are at least partially identical to the 5′- and 3′-ends of the HIV-1 quasi-species LTR regions. In yet other embodiments, the gRNAs are unique to the HIV-1 virus. It is preferred that the gRNAs are not homologous to the host human genome, as to limit the likelihood of undesired manipulations of the human's genome.

The guide RNA also comprises a second region that forms a secondary structure. In certain embodiments, the secondary structure comprises a stem (or hairpin) and a loop. The length of the loop and the stem can vary. For example, the loop can range from about 3 to about 10 nucleotides in length, and the stem can range from about 6 to about 20 base pairs in length. The stem can comprise one or more bulges of 1 to about 10 nucleotides. Thus, the overall length of the second region can range from about 16 to about 60 nucleotides in length. In an exemplary embodiment, the loop is about 4 nucleotides in length and the stem comprises about 12 base pairs.

The guide RNA also comprises a third region at the 3′ end that remains essentially single-stranded. Thus, the third region has no complementarity to any chromosomal sequence in the cell of interest and has no complementarity to the rest of the guide RNA. The length of the third region can vary. In general, the third region is more than about 4 nucleotides in length. For example, the length of the third region can range from about 5 to about 30 nucleotides in length.

In other embodiments, the guide RNA can comprise two separate molecules. The first RNA molecule can comprise the first region of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in certain embodiments, the first and second RNA molecules each comprise a sequence (of about 6 to about 20 nucleotides) that base pairs to the other sequence. In the embodiments where the guide RNA is introduced into the cell as a DNA molecule, the guide RNA coding sequence can be operably linked to promoter control sequence for expression of the guide RNA in the eukaryotic cell. For example, the RNA coding sequence can be operably linked to a promoter sequence that is recognized by RNA polymerase III (Pol III). Examples of suitable Pol III promoters include, but are not limited to, mammalian U6 or H1 promoters. In exemplary embodiments, the RNA coding sequence is linked to a mouse or human U6 promoter. In other exemplary embodiments, the RNA coding sequence is linked to a mouse or human H1 promoter. The DNA molecule encoding the guide RNA can be linear or circular.

In certain embodiments, the DNA sequence encoding the guide RNA can be part of a vector. Suitable vectors include plasmid vectors, phagemids, cosmids, artificial/mini-chromosomes, transposons, and viral vectors. In an exemplary embodiment, the DNA encoding the RNA-guided endonuclease is present in a plasmid vector. Non-limiting examples of suitable plasmid vectors include pUC, pBR322, pET, pBluscript, and variants thereof. The vector can comprise additional expression control sequences (e.g., enhancer sequences, Kozak sequences, polyadenylation sequences, transcriptional termination sequences, and so forth), selectable marker sequences (e.g., antibiotic resistance genes), origins of replication, and the like.

In certain embodiments, the number of gRNAs useful within the present invention ranges from about 1 to about 100, about 1 to about 75, about 1 to about 50, or about 1 to about 25. In other embodiments, the number of gRNAs ranges from about 1 to 10. In yet other embodiments, the number of gRNAs ranges from about 1 to 8. In further embodiments, the number of gRNAs ranges from 1 to 4 (exp 23) (i.e., the total number of possible 23-mers).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in US20110059502, incorporated herein by reference. In certain embodiments, a tagged CRISPR enzyme is used to identify the location of a target sequence.

In certain embodiments, a CRISPR enzyme in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome, exosomes or any other membrane-derived membrane encased delivery system, or nanoparticle. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

The complex CRISPR/Cas proteins can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3,Csx17, Csx14, Csx10, Csx16, CsaX, Csx3, Csz1, Csx15, Csf1, Csf2, Csf3, Csf4, and Cu1966. In certain embodiments, one or more elements of a CRISPR system is derived from a type I, type II, or type III CRISPR system.

In certain embodiments, the CRISPR/Cas is comprises a Type II CRISPR/Cas system. The Type II CRISPR/Cas sytem utilizes the Cas9 endonuclease protein. The Cas9 protein can be from Streptococcus pyogenes, Streptococcus thermophilus, Streptococcus mutans, Neisseria meningitidis, or other bacterial or archaeal species. Cas9 is guided by a mature crRNA that contains a spacer sequence, about 20 base pairs of target sequence, and a trans-activated small RNA (tracrRNA). The tracrRNA:crRNA duplex directs Cas9 to target DNA via complementary base pairing between the spacer and the complementary sequence (the protospacer) on the target DNA. Cas9 recognizes a trinucleotide (NGG) protospacer adjacent motif (PAM) to specify the cut site (the 3rd nucleotide from the PAM). The crRNA and tracr RNA can be expressed separately or engineered into an artificial fusion small guide RNA (sgRNA) via a synthetic stem loop to mimick the natural crRNA/tracrRNA duplex. Such sgRNA can be synthesized or in vitro transcribed for direct RNA transfection, or expressed from a promoter-driven RNA expression vector.

In general, CRISPR/Cas proteins comprise at least one RNA recognition and/or RNA binding domain. RNA recognition and/or RNA binding domains interact with the guiding RNA. CRISPR/Cas proteins can also comprise nuclease domains (i.e., DNase or RNase domains), DNA binding domains, helicase domains, RNAse domains, protein-protein interaction domains, dimerization domains, as well as other domains. The CRISPR/Cas proteins can be modified to increase nucleic acid binding affinity and/or specificity, alter an enzymatic activity, and/or change another property of the protein. In certain embodiments, the CRISPR/Cas-like protein of the fusion protein can be derived from a wild type Cas9 protein or fragment thereof. In other embodiments, the CRISPR/Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In other embodiments, the Cas9 nucleic acid sequence can be codon optimized for efficient expression in mammalian cells, i.e. “humanized”. A humanized Cas9 nuclease sequence can be encoded by any of the expression vectors listed in Genbank accession numbers KM099231.1 GI:669193757; KM099232.1, GI669193761; or KM099233.1 GI669193765. The Cas9 nuclease can also be contained within a commercially available vector such as PX330 or PX260 from Addgene (Cambridge, Mass.). In certain embodiments, the Cas9 endonuclease can have an amino acid sequence that is a variant or a fragment of any of the Cas9 endonuclease sequences of Genbank accession numbers KM099231.1 GI:669193757; KM099232.1, GI669193761; or KM099233.1 GI669193765 or Cas9 amino acid sequences of PX330 or PX260 from Addgene (Cambridge, Mass.). In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art. In certain embodiments, the endonuclease sequence is optimized for expression in a human cell.

The present invention also includes a vector driving the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook, et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In certain embodiments, adenovirus vectors are used. In certain embodiments, lentivirus vectors are used.

In certain embodiments, the invention contemplates the use of nanoparticles for the delivery of the gRNAs contemplated herein. In other embodiments, the nanoparticles allowed for the delivery of the gRNAs to the nucleus of a cell. In yet other embodiments, the nanoparticles comprise a targeting agent that directs it to the cell and/or the nucleus thereof.

In yet other embodiments, the nanoparticles are biodegradable. In yet other embodiments, the nanoparticles of interest, as well as other delivery vehicles of interest, are illustrated in one or more of the following publications, all of which are incorporated herein by reference in their entireties: WO 2015127437, WO 2015108945, WO 2014169207, WO 2014085795, WO 2013159092, WO 2013158549, WO 2012061480, WO 2009111638, WO 2008141155, WO 2007124224, WO 2004112747, US 20150079007, US 20150297587, US 20130236553, US 20050048002, US 20060280430, US 20090274765, US 20100291065, US 20150125401, and EP2259798.

In certain embodiments, the HIV-1-infected human is treated by antiretroviral therapy (ART) or the highly active antiretroviral therapy (HAART). In certain embodiments, the HIV-1 excision with the CRISPR/Cas9 system is performed on a human that has received, is receiving or will receive ART/HAART treatment. In certain embodiments, the ART/HAART treatment is continued, modified or terminated after HIV-1 excision with the CRISPR/Cas9 system.

In certain embodiments, the HIV-1 LTR regions are sequenced at two or more time points, and the number of HIV-1 vQS infecting the human is estimated at each time point. In other embodiments, the CRISPR/Cas9 excision step is performed when the number of vQS is minimized or has reached a minimum over the sequencing time period. In yet other embodiments, the CRISPR/Cas9 excision step is repeated until complete HIV eradication.

In certain embodiments, a composition of isolated set of guide RNAs (gRNAs) is provided. In other embodiments, the gRNAs set of this invention comprises gRNAs that are at least partially identical to a fragment of the HIV-1 LTR region or any other HIV-1 genome region that is integrated in the genomic DNA of an HIV-1-infected human.

In certain embodiments, the level of identity between the gRNA and its targeted HIV-1 LTR region is determined by the degree of complementarity. In other embodiments, the degree of complementarity between a gRNA and its corresponding target sequence, when optimally aligned using a suitable alignment algorithm, is about or more than about 50%, 60%, 75%, 80%, 85%, 90%, 95%, 97.5%, 99%, or more. In yet other embodiments, the fragment of the HIV-1 LTR regions is selected from at least one from the group consisting of the 5′-end and 3′-end. In yet other embodiments, the human is infected with one or more evolving HIV-1 quasi-species (vQS). In yet other embodiments, the vQS comprise one or more viral single nucleotide polymorphism (vSNP) in the integrated HIV-1 LRT nucleotide sequence, as compared to the HIV-1 LTR nucleotide sequence from an HIV-1 consensus strain. In yet other embodiments, the HIV-1 consensus strain is from the subtype B.

In certain embodiments, the vSNP can occur at various location within the HIV-1 LTR. In other embodiments, the vSNP is present at the nucleotide position selected from the group consisting of 1-800.

In certain embodiments, hardware or one or more software or programs or packages can be utilized to define an HIV population or identify in which HIV population a subject belongs. A next-generation sequencer such those available from Illumina, Inc. of San Diego, Calif. or Pacific Biosciences of California, Inc. of Menlo Park, Calif. can be used to sequence DNA isolated from cells obtained from a subject, some of these cells will include chromosomally integrated copies of the HIV-1 genome. The next generation sequencer produces a file that includes a large list of sequence reads from the subject.

This file is then imported into an alignment program that aligns the reads from the file to an HIV-1 reference sequence. Suitable alignment programs include the Burrows-Wheeler Aligner available at http://bio-bwa dot sourceforge dot net/, the LASTZ aligner available from Pennsylvania State University of University Park, Pa., and the SEGEMEHL aligner available from Leipzig University of Leipzig, Germany at www dot bioinf dot uni-leipzig dot de/Software/segemehl/. The alignment program produces a computer-readable BAM (Binary Alignment Format) file indicating regions of alignment.

These aligned reads were then put into the program using the BioPython library. Further, many similar tools exist that provide this same functionality. In certain embodiments, once in the program the aligned reads are represented as strings of letters corresponding to the nucleotides of the sequence read. The reverse complement of the read is also added to account for gRNAs found on the opposite strand.

In order to gather all potential gRNA hits any 23-mer ending in GG from the read is collected into a tab-delimited file for later processing. In certain embodiments, this size range can be varied between 5 and 1,000. If the methodology is being applied to many biological samples then the potential gRNAs are collated to keep only a single instance of each unique gRNA for downstream consideration. In certain embodiments, these potential gRNAs are filtered to only keep the most common potential gRNAs to reduce processing time. In other embodiments, a greater sensitivity is desired and all potential gRNAs are kept.

Any list of potential gRNAs can be compared back to a set of samples to assess the in silico likelihood of cleavage across the entire vQS. This is accomplished by reading in the sequences as before and comparing each potential gRNA against the read using a binding matrix. In some embodiments this is done using a binding matrix described by Hsu et al., 2013, Nature Biotech. 31:827-832, which assigns a position specific penalty for a mismatch between the gRNA and the read. The weighted penalties are then summed to get a likelihood that the gRNA causes a cleavage at that specific location. The speed of this process is improved by only looking at positions that end in a GG. In some embodiments this can also take into account the quality of the NGS base-call at the positions as well.

Once each gRNA has been associated with each read, one can calculate statistics about how the list of gRNAs binds with the vQS. This includes: the average likelihood to cut the vQS at each position, the number of cuts that occurs on any specific strand or the number of strands that are uncut. These values can be used to optimize the package of gRNAs for downstream use.

In some embodiments in which the binding matrix is similar to that described by Hsu, et al., 2013, Nature Biotech. 31:827-832 (as well as any other binding matrices with the same properties), the position specific penalty matrix can be run on a summary of the reads instead of the individual reads. This is done by finding all nucleotides that occur at each position of the genome as well as their relative fractions. This can be done by computationally processing the alignments to count their relative frequencies. With the relative frequencies at each position calculated, the binding matrix from Hsu, et al., 2013, Nature Biotech. 31:827-832 can be applied to determine the fraction of reads at any particular position that is cleaved by a given gRNA.

The methodology can also be used to pick a collection of gRNAs that affect the largest portion of the vQS. The size of the collection can range from 2 to infinity. This is done by first picking and optimizing all potential gRNAs and then measuring their individual binding effectiveness. The ability of the package, as a whole, to cleave the vQS can be mathematically described by using a statistical OR function. This measures the likelihood that a strand is cleaved by any of the gRNAs in the package assuming a random distribution of their binding. A statistical OR is the 1 minus the product of the binding efficiency of the individual gRNAs. With that information, numerical optimization can be used to find the minimal set of gRNAs that effect the largest portion of the QS.

Furthermore this methodology can be used to pick a collection of guide sequences that affect the largest portion of the vQS of a collection of samples. This is done by first picking the collection of guide sequences for each sample as discussed previously. All of these potential gRNAs are then reapplied to every other sample. Numerical optimization techniques can then be used to pick the optimal set of gRNAs from this collection. This can be trivially expanded to measuring the effectiveness of a set of guide sequences on the collection of samples.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### General Materials and Methods

### Patient Enrollment, Clinical Data, and Sample Collection:

Patients in the Drexel Medicine CARES Cohort were recruited under a protocol adhering to the ethical standards of the Helsinki Declaration (Li, et al., 2011, J. Neurovirol. 17:92-109). All patients provided written consent upon enrollment. Patients were called back for longitudinal study approximately every 6 months, with at least one recall per year.

### Peripheral Blood Mononuclear Cell Isolation:

At each 6-month visit, blood was collected. One gray-top tube was sent for drugs-of-abuse screening (˜10 mL) and four purple-top BD vacutainer tubes (Becton Dickinson & Co., Franklin Lakes, N.J.) containing EDTA were used to collect blood from patients (˜40 mL) for serum and PBMC isolation, as described (Li, et al., 2011, J. Neurovirol. 17:92-109). From 5×10⁶ PBMCs, genomic DNA and total RNA isolation was performed using a Qiagen (Venlo, Limburg, Netherlands) AllPrep DNA/RNA procedure.

Additional Materials and Methods specific for Example 1
(a) PCR Amplification and Sequencing of the HIV-1 LTR from Patient Genomic DNA:

From the genomic DNA, PCR was performed to amplify and sequence the HIV-1 LTR (Li, et al., 2011, J. Neurovirol. 17:92-109). Analysis of RNA viral genomes for evidence of genetic variation within the genome may be affected by polymerase selection (Bracho, et al., 1998, J. Gen. Virol. 79 (Pt 12):2921-8). Taq polymerase created variants as a result of the polymerase rather than naturally occurring within the genome. Pfu DNA polymerase showed a greater fidelity and did not introduce false positive sequence variants. The studies presented here used the Phusion DNA polymerase that features an error rate 6-fold less than Pfu DNA polymerase. In certain embodiments, all variants seen in this study were the result of changes inserted by the patient's viral polymerase and not a result of the amplification process. A subset of 10 patient samples were deep-sequenced to confirm that the PCR technique captured the predominant viral quasi-species.

### (b) Analysis of Sequencing Results:

The overall LTR sequence for each patient was analyzed for sequence variation throughout the entire LTR as described (Li, et al., 2011, J. Neurovirol. 17:92-109). Sequences were aligned to the Consensus B (January 2002) reference sequence (Los Alamos HIV-1 Sequence Database. Available at www dot hiv dot lanl dotgov, accessed Sep. 1, 2006). Both quality information from the trace files (PHRED scores (Brockman, et al., 2008, Genome Res 18:763-70), and several statistical tests for identification and quality control of the called putative variations were used to identify high-quality SNPs. The Neighborhood Quality Standard (NQS) method of Altshuler and Brockman was used for SNP calling and validation (Brockman, et al., 2008, Genome Res 18:763-70; Altshuler, et al., 2000, Nature 407:513-6). Final sequences have been submitted to Genbank: temporary ID grp-4607538.

### Example 1

### HIV Quasi-Species Excision Utilizing CRISPR/Cas9 Technology

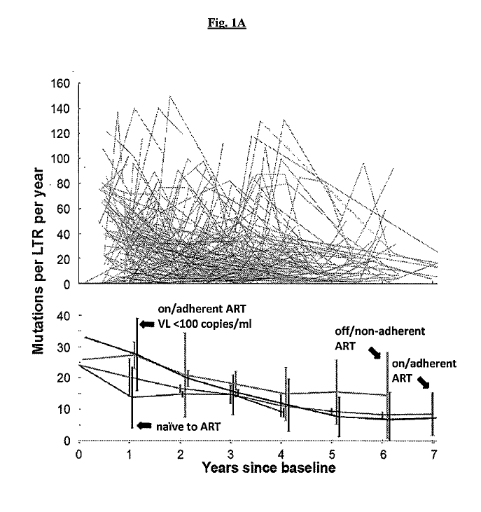

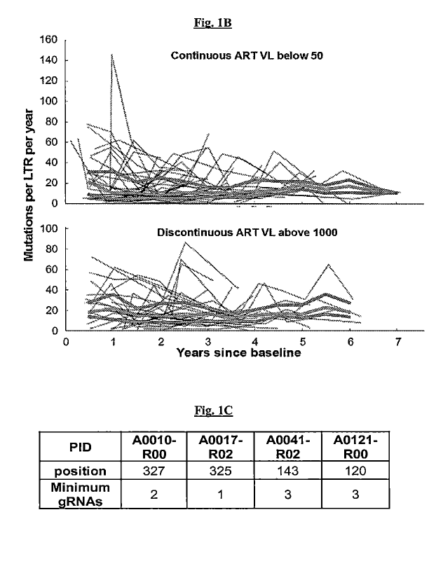

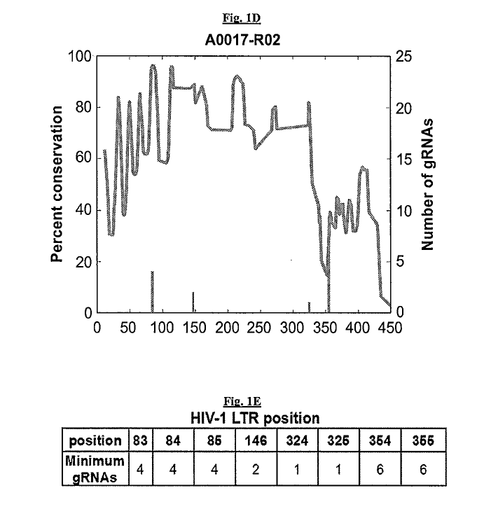

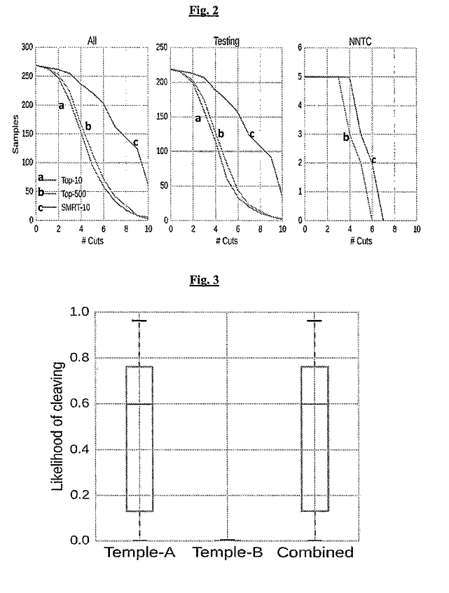

The sequence analyses from HIV-1-infected patients enrolled in the Drexel Medicine CNS AIDS Research and Eradication Study (CARES) Cohort (Li, et al., 2011, J. Neurovirol. 17:92-109) showed that the predominant sequence of the LTR from integrated provirus from PBMCs exhibited a decrease in the number of variations per year regardless of type of therapy (FIG. 1A). However, the virus still underwent continued genetic change of the predominant genotype in these cells for at least 6 years while on effective suppressive ART, with a constant median of 10-20 unique mutations per year throughout the entire LTR (FIG. 1B).

Given these results, the use of next-generation sequencing is essential to determine all of the viral quasi-species (vQS) present in a well-controlled patient's reservoir. This approach allows designing gRNA regimen that eliminate, with excision therapy, all vQS present. This eliminates the need to understand if a given infected cell has a replication competent virus or not, as it eliminates all HIV-1 targets present within a given cell population.

Roche 454 next generation sequencing (NGS) was performed on genomic DNA isolated from PBMCs of 6 patients and 8 samples to gain an appreciation for the conservation of the HIV-1 LTR and number of gRNAs potentially needed per patient to target all known quasi-species. Due to the limitation of gRNA construction, a complete gRNA regimen could only be designed for 4 of the 8 patient samples (FIG. 1C). Within these samples, none needed more than ten gRNAs to target the entire quasi-species in a given patient. This showed that, for a subset of patients, even those with viral loads above 50 copies/mL, a regimen with less than ten gRNAs can be devised. Conversely, for another subset of patients, even if they have viral loads below 50 copies/mL, no regimen can completely excise their infection. Of the NGS samples, 2 of the 6 patients were tested longitudinally 11 months apart. When looking at longitudinal samples from these patients, the amount of variation in the LTR becomes more conserved (however not lacking of genetic variation) across the entire sequence.

One of these patients, patient A0017, was further evaluated. At the intake visit, at which point patients had been on HAART for six years, patient A0017 could not have been completely treated with CRISPR/Cas9 excision therapy. Over the course of another year of HAART therapy, it was possible to design an excision regimen of less than 10 gRNAs at various sites to be able to cover all the known quasi-species from the PBMC population (FIGS. 1D-E). In certain aspects, this indicates that the vQS is a moving target and the therapeutic window is limited for a given set of gRNAs even with the most effective therapies currently available.

### Additional Materials and Methods Specific for Examples 2-4

Post PCR-amplification, the Nextera XT DNA Library Preparation Guide (Illumina Cat #FC-131-1096) was followed, per manufacturer instructions, except where specified. Amount of input DNA was changed from 0.2 ng/μL to 0.4 ng/μL. Tagmentation reaction was shortened from 5 minutes at 55° C. to 4 minutes and 30 seconds to avoid over-fragmenting the shorter PCR fragments. PCR clean-up was completed using 90 μL, per sample of AxyPrep™ Mag PCR Clean-Up Kit (Axygen® Cat# MAG-PCR-CL-5). Libraries were normalized using the Quant-iT™ dsDNA Assay Kit, High Sensitivity (Invitrogen Cat# Q33120), and validated on an Agilent Technology 2100 Bioanalyzer using the Agilent High Sensitivity DNA Kit (Agilent Technologies Cat# 5067-4626). Based on the average size of 500 bp, libraries were clustered using the equation 1 ng/μl=3 nM. Samples were pooled together to reach a final concentration of 1 nM, and sequenced using the NextSeq® 500/550 Mid Output Reagent Cartridge v2 300 cycles (Illumina Cat#FC-404-2003) on the Illumina NextSeq 500 Desktop Sequencer.

gRNA Prediction:

NGS sequences were aligned to the HIV-1 genome using the BWA alignment tool. These aligned reads were then imported into the program using the BioPython library. Once in the program, they were represented as strings of letters corresponding to the nucleotides of the sequence read. The reverse complement of the read was also added to account for gRNAs found on the opposite strand.

In order to gather all potential gRNA hits any 23-mer ending in GG from the read were collected into a tab-delimited file for later processing. This produced anywhere from 0 to 1.2 million possible gRNAs per biological sample, and the potential gRNAs were collated to keep only a single instance of each unique gRNA for downstream consideration. For this analysis only the top 10,000 most common gRNAs were kept for downstream

The list of potential gRNAs from 100 randomly chosen samples were compared back to those same set of samples to assess the in silico likelihood of cleavage across the entire vQS for each sample and each gRNA. This was accomplished by reading in the sequences as before and comparing each potential gRNA against the read using the binding matrix described by Hsu, et al., 2013, Nature Biotech. 31:827-832. This assigned a predicted likelihood of cleavage for each read that was averaged over all reads which covered the predicted position.

Two methods of selected packages of gRNAs were tested from this set of 100 samples. Two of the formulations were taken as simply the top 10 and top 500 gRNAs with respect to their complementarity to the sample QS in the training set. A third package was selected using a simple numerical optimization technique by first starting with the gRNA with the highest complementarity and then iteratively choosing gRNAs that have the highest complementarity to the samples that were missed by the previously selected gRNAs. This is termed the SMRT-10 package of gRNAs.

### Example 2

### Designing gRNAs from the Drexel Medicine CARES Cohort Patients and Measuring their Effectiveness on Additional CARES Cohort Patient Samples and Samples from the National NeuroAlDS Tissue Consortium (NNTC) Texas Collection Site

Illumina next generation sequencing (NGS) was performed on genomic DNA isolated from PBMCs of 264 samples from 168 unique patients in the Drexel Medicine CARES cohort and 5 samples from 3 patients in the NNTC cohort to gain an appreciation for the conservation of the HIV-1 LTR and number of gRNAs potentially needed to target all known quasi-species. From the 264 PBMC samples, 100 were randomly selected to comprise the training dataset and the other 164 were held out to function as a testing dataset.

Solely using data from the 100 training samples gRNAs were determined by finding all segments of the LTR sequences ending in GG resulting in over 4.65 million possibilities. Each of these were then rescanned against the 100 training samples to determine the level of identity between each gRNA and each quasi-species in the sample. From this data multiple formulations were created to account for the many potential delivery systems for the gRNA excision therapy.

Two of the formulations were taken as simply the top 10 and top 500 gRNAs with respect to their complementarity to the sample QS in the training set. A third package was selected using a simple numerical optimization technique by first starting with the gRNA with the highest complementarity and then iteratively choosing gRNAs that have the highest complementarity to the samples that were missed by the previously selected gRNAs. These gRNAs as well as their effectiveness are shown in FIG. 5. These four packages were then compared to the entire dataset (FIG. 2A), the held-out dataset (FIG. 2B) and the independent dataset (FIG. 2C).

These experiments indicated that, out of the 164 samples held-out, only 2 samples were missed by the potential packages. Furthermore, on an independent set of samples (N=5) from another the NNTC Texas collection site cohort, no patients were missed by the potential packages. Not only are these patient samples from a different location in the United States showing the potential for a broad design, these samples were also from the brain and spleen, making these gRNA packages even broader with respect to the potential of them to excise HIV-1 in many anatomical locations within a patient.

### Example 3

### Measuring the Effectiveness of Previously Described gRNAs Against the Drexel Medicine CARES Cohort Patient Samples

Assessing how well a package of gRNAs cleaves a set of patients is one of the first steps associated with determining its effectiveness as a potential therapy. In order to do this two gRNAs from a previous study (termed A and B; Hu, et al., 2014, Proc. Nat. Acad. Sci. USA 111(31):11461-11466, see also PCT/US2014/053441) were compared to the patient samples from the Drexel Medicine CARES cohort. The computational evaluation was performed using the gRNAs A and B as inputs and compared to the 269 NGS samples as described previously.

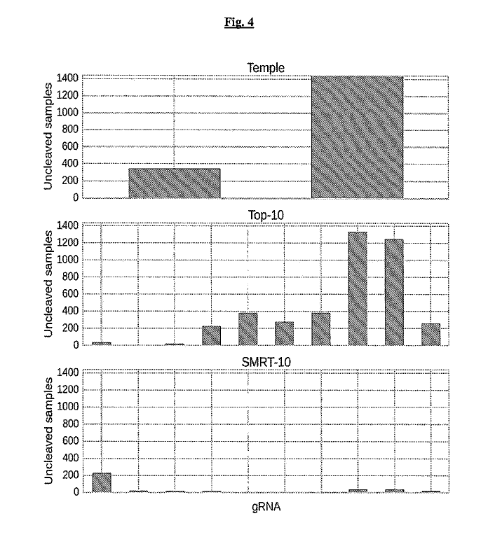

NGS reads were aligned to the HXB2 genome using the BWA aligner and those falling within the LTR region were kept. Those reads that overlap the regions expected to be cut by gRNAs A and B were checked for cleavage efficiency using the method described in Hsu, et al., 2013, Nature Biotech. 31:827-832. The fraction of reads having at least an 80% were then calculated for each sample. FIG. 3 shows a boxplot indicating the spread of binding efficiencies for each individual gRNA and the package taken as a whole.

### Example 4

### Measuring the Effectiveness of the CARES Cohort gRNA Packages on HIV-1 Sequences Obtained from the Los Alamos National Laboratory (LANL) HIV Sequence Database

In order to assess whether a set of gRNAs will work on a large collection of patients, in silico screening may be used to ensure a broad-spectrum package. While all of the previous examples used NGS data, this one uses data from Sanger sequences submitted to the LANL database. In order to perform these studies, all North American Subtype B HIV-1 sequences were downloaded from LANL (downloaded as of Oct 1 2014). These sequences were then aligned to the HIV reference genome (HXB2), and only those that overlapped the LTR region of HIV-1 were kept. This resulted in a total of 1,471 sequences. The gRNAs that were determined above in Example 2 (FIG. 5 and FIG. 2) as well as gRNAs A and B from Hu, et al., 2014, Proc. Nat. Acad. Sci. USA 111(31):11461-11466 were compared to the LANL sequences.

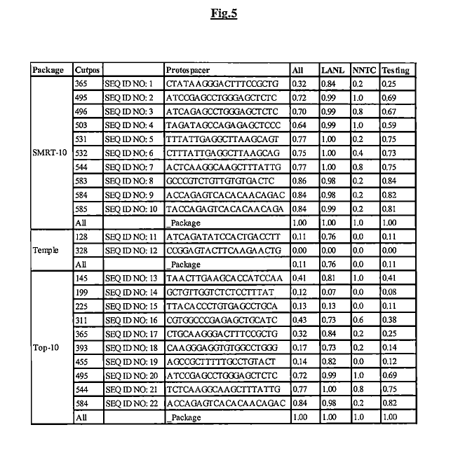

FIG. 4 shows the number of samples with a likelihood of cleavage less than 80% for the two gRNAs from Temple (top panel), the top-10 gRNAs measured by cleavage efficiency (middle panel), and the SMRT-10 gRNAS (bottom panel). Furthermore, using this data it was shown that, with the Top-10 and SMRT-10 packages, each sample was cleaved at least once with an average of 1.3 and 5.4 times respectively.

This demonstrates the broad ability of gRNAs designed in this way and their potential to cleave all known subtype B sequences from across North America. Taken together, these results show that NGS techniques allow identifying all quasi-species present in cell populations and show that the number of gRNAs needed is low enough to be packaged into delivery systems such as any of a number viral vectoring or nanoparticle strategies. Taken together, the methodologies described above produce a package of gRNAs that will be much more efficient at removing HIV-1 than previously described methods.

The design of the gRNAs necessary for the excision of all integrated virus and the efficient delivery of the gRNAs are important factors for CRISPR/Cas9 system. Delivery can be achieved for example using lentiviral vectors. This type of delivery system favors the infection of similar cell types that HIV naturally infects, potentially limiting the delivery of the therapy to uninfected off target cells. With respect to the viral genotype, the genetic makeup of the vQS retained in reservoir cells like the CD4 memory T-cell population is determined and compared to the vQS retained in cells of the monocyte-macrophage lineage, in order to define the differences with respect to viral eradication involving LTR region targeting. In non-limiting embodiments, deep sequencing studies in well-defined patient populations is performed using long fragment PCR techniques for proper gRNA design. These studies allow delimiting viral regions of high conservation, even across multiple tissues. Use of deep sequencing allows for defining the viral dynamics in these cells, and for determining the length of an ART that is required to drive virus selection in the reservoir to a level where the number of quasi-species present is low enough to limit the gRNAs needed to eradicate the viral proviral DNA in these tissues. Sequencing of patients, especially at cell subpopulation levels, is critical to have a complete understanding of viral dynamics in well-controlled patients. This challenge is addressed by advances in the delivery of the CRISPR/Cas9 system allowing larger cassettes of gRNAs to overcome the variability observed even after prolonged therapy.

In certain embodiments, ex vivo proof-of-concept studies beginning with the memory T-cell population determine if virus can be eradicated from HIV-1-infected patient derived cells in a well-controlled experimental environment within single cell T-cell populations cultured in vitro from HIV-1-infected patients.

The excision approach of the present invention represents the only therapeutic strategy that can achieve complete elimination of competent and non-competent proviruses. Thus, the compositions and methods disclosed in this invention allow HIV eradication for an HIV-1-infected human.

The disclosures of each and every patent, patent application, and publication cited herein are hereby incorporated herein by reference in their entirety.

While the invention has been disclosed with reference to specific embodiments, it is apparent that others skilled in the art may devise other embodiments and variations of this invention without departing from the true spirit and scope of the invention. The appended claims are intended to be construed to include all such embodiments and equivalent variations.

1. A method of identifying one or more guide RNAs (gRNAs) that affect clustered regularly interspaced short palindromic repeats (CRISPR)/CRISPR associated (Cas)9 system-mediated cleavage of a viral genomic region that is chromosomally integrated in a subject infected with the virus, wherein the virus comprises one or more virus quasi-species (vQS), the method comprising:
sequencing the test genomic material isolated from a bodily sample selected from the group consisting of a bodily sample of the virus-infected subject and bodily samples from a virus-infected patient population,
identifying a given set of candidate gRNAs that aligns to the consensus sequence of the reference virus, and
comparing the given set of candidate gRNAs to the sequence of the test genomic material,
thus assessing whether each candidate gRNA in the given set affects CRISPR/Cas9-mediated cleavage of the viral genomic region that is chromosomally integrated in the virus-infected subject.

2. The method of claim 1, wherein the virus comprises a lentivirus or retrovirus.

3. The method of claim 2, wherein the virus comprises HIV-1 or HIV-2.

4. (canceled)

5. The method of claim 1, wherein the viral genomic region comprises an HIV-1 integrated long terminal repeat (LTR) region of at least one vQS.

6. (canceled)

7. The method of claim 1, further comprising counting the number of individual instances of alignment between each candidate gRNA and the sequence of the test genomic material, further comprising selecting a group of candidate gRNAs that have the highest number of individual instances of alignment for use in the comparing step, wherein the group comprises at least one gRNA.

8. (canceled)

9. The method of claim 1, wherein the comparing step comprises applying a binding matrix, which assigns a position-specific penalty for a mismatch between each candidate gRNA and the sequence of the test genomic material.

10-13. (canceled)

14. A tangible, non-transitory computer-readable medium comprising computer-executable instructions for implementing the method of claim 1.

15. A method of treating HIV-1 infection in an infected human, the method comprising the steps of:
sequencing HIV-1 long terminal repeat (LTR) regions that are integrated in the human genomic DNA from a sample selected from the group consisting of a bodily sample from the HIV-1-infected human or bodily samples from a HIV-1-infected patient population;
identifying a set of guide RNA sequences (gRNAs) that are at least partially identical to a fragment of the HIV-1 LTR regions; and,
excising the HIV-1 chromosomally integrated genome from the human genomic DNA of the human using the set of gRNAs and the clustered regularly interspaced short palindromic repeats (CRISPR)/CRISPR associated (Cas)9 system.

16. The method of claim 15, wherein the sequencing comprises next-generation sequencing.

17-18. (canceled)

19. The method of claim 15, wherein the human is infected with one or more evolving HIV-1 quasi-species (vQS).

20. The method of claim 19, wherein the vQS comprise one or more viral single nucleotide polymorphism (vSNP) in the integrated HIV-1 LTR nucleotide sequence, as compared to the HIV-1 LTR nucleotide sequence from an HIV-1 reference/consensus strain.

21. (canceled)

22. The method of claim 19, wherein the gRNAs are at least partially identical to the 5′- and 3′-ends of the HIV-1 vQS LTR regions.

23. (canceled)

24. The method of claim 15, wherein the human is being administered HAART.

25. The method of claim 19, wherein the sequencing is repeated at two or more time points, and the number of HIV-1 vQS infecting the human is estimated at each time point, wherein the excision step is performed when the number of vQS is minimized or has reached a minimum over the sequencing time period.

26-27. (canceled)

28. The method of claim 15, wherein the excision step comprises administering to the human the set of gRNAs within at least one selected from the group consisting of a viral vector, microparticle, nanoparticle, liposome, hydrogel, and block copolymer micelle.

29-30. (canceled)

31. The method of claim 20, wherein the HIV-1 consensus strain is from the subtype B.

32. (canceled)

33. A method of treating HIV-1 infection in an infected human, the method comprising the steps of:
obtaining a set of guide RNA sequences (gRNAs) that are at least partially identical to a fragment of the HIV-1 LTR regions; and,
excising the HIV-1 genome from the genomic DNA of the human using the set of gRNAs targeted to the HIV-1 LTR or another region of the HIV-1 and the CRISPR-Cas9 system.

34. An isolated set of guide RNAs (gRNAs), the set comprising gRNAs that are at least partially identical to a fragment of the HIV-1 LTR regions that are integrated in the genomic DNA of an HIV-1-infected human.

35. (canceled)

36. The isolated set of claim 34, wherein the human is infected with one or more HIV-1 quasi-species (vQS).

37. The isolated set of claim 36, wherein the vQS comprise one or more viral single nucleotide polymorphism (vSNP) in the integrated HIV-1 LTR nucleotide sequence, as compared to the HIV-1 LTR nucleotide sequence from an HIV-1 reference/consensus strain.

38. The isolated set of claim 37, wherein the HIV-1 consensus strain is from the subtype B.

39. The isolated set of claim 36, wherein the HIV-1 vQS latently infect at least one host cell selected from the group consisting of a macrophage, gut-associated lymphoid cell, microglial cell, astrocyte, and resting CD4+ memory T-cell.

40. The isolated set of claim 36, wherein the gRNAs are at least partially identical to the 5′- and 3′-ends of the HIV-1 vQS LTR regions.

41-45. (canceled)

46. The isolated set of claim 34, which comprises at least one gRNA encoded by a DNA sequence selected from the group consisting of SEQ ID NOs:1-10 and SEQ ID NOs:13-22.

47-48.(canceled)

49. A method of defining an HIV population or identifying to which HIV population a human subject belongs, the method comprising:
obtaining a DNA sequencing file for the human subject, wherein the file comprises an ordered sequence of bases corresponding to the subject's DNA;
using an alignment program to align the human subject's DNA sequence with a reference sequence, whereby the program generates a computer-readable VCF (Variant Call Format) file indicating regions of alignment; and,
analyzing differences of regions of human subject's DNA as compared to the reference sequence;
wherein the analysis allows for defining an HIV population or identifying to which HIV population a human subject belongs.
